(19) United States
(12) Patent Application Publication
(10) Pub. No.: US 2012/0094985 A1
(43) Pub. Date: Apr. 19, 2012

(54) PHARMACEUTICAL COMPOSITIONS COMPRISING CHLOROPHENYL PIPERAZINE DERIVED COMPOUNDS AND USE OF THE COMPOUNDS IN PRODUCING MEDICAMENTS

(75) Inventor: Ing-Jun Chen, Kaohsiung (TW)

(73) Assignee: KAOHSIUNG MEDICAL UNIVERSITY, Kaohsiung City (TW)

(21) Appl. No.: 13/340,041

(22) Filed: Dec. 29, 2011

Related U.S. Application Data
(62) Division of application No. 12/620,448, filed on Nov. 17, 2009.

(30) Foreign Application Priority Data
Apr. 30, 2009 (TW) 098114472

(51) Int. Cl. A61K 31/496; A61K 31/5415; A61P 25/24

(52) U.S. Cl. 514/224.8; 514/252.16

(57) ABSTRACT

The invention relates to a pharmaceutical composition for treating serotonergic neurotransmission related disease or condition, including an effective amount of a chlorophenylpiperazine derived compound of Formula (I),

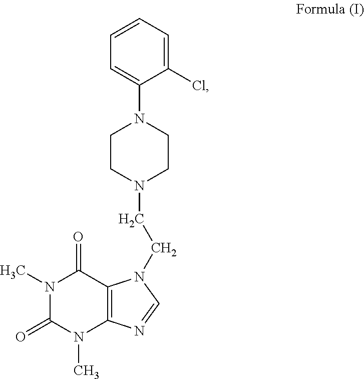

or the pharmaceutically acceptable salt thereof. The pharmaceutical composition is safe and does not have side effect on lethargy.

### CROSS REFERENCE TO RELATED APPLICATION

This application is a divisional of U.S. patent application Ser. No. 12/620,448, filed Nov. 17, 2009, which claims priority to TW 098114472, filed Apr. 30, 2009, both of which are incorporated herein by reference as if fully set forth.

### FIELD OF THE INVENTION

The present invention relates to a pharmaceutical composition. In particular, the present invention relates to a pharmaceutical composition for treating serotonergic neurotransmission-related diseases or illness. The present invention also relates to an use of a chlorophenyl piperazine derivative. In particular, the present invention relates to an use of the chlorophenyl piperazine derivative on manufacturing pharmaceuticals.

### BACKGROUND OF THE INVENTION

Modern people face incrementally increasing pressure and depression patients gradually increase along with civilization development. Their living quality is not only effected, but also results in social problems. Thus, plenty of antidepressants are continuously developed, but most have harmful side effects and safety is suspicious. Therefore, many harmful hepatotoxic antidepressants, such as Benoxaprofen, Bromofenac, Tasosartan, Pemoline, Troglitazone, Nefazodone, and so on, are continually forbidden by countries.

Trazodone (referring to U.S. Pat. No. 3,381,009) is a common antidepressant prescription. Both Trazodone and Nefazodone (referring to U.S. Pat. No. 4,338,317) have a structure of 3-chlorophenyl piperazine and metabolize as a structure of meta-chlorophenylpiperazine (m-CPP) to have anti-histaminergic activity. Trazodone has been widely used for thirty years because of its relative safety. However, patients' daily lives are affected and their quality of life cannot be taken into consideration since Trazodone and Nefazodone has significant side effect on lethargy.

Generally, it is proposed that a structure of chlorophenyl piperazine has serotonin-regulated mechanism. In addition to m-CPP, it is also proposed that ortho-chlorophenylpiperazine (o-CPP) might be related to antagonism of serotonin receptor. However, there is no pharmaceutical having such chemical structure marketed, and the potential of this kind of drug is unknown.

At present, the pharmacological mechanism for anti-depression is illustrated as follows.

Nitric oxide (NO) plays an important role in the control of behavior (Reddy et al., 1998) as well as of locomotion. Alternations in NO synthase (NOS) activity and/or NO concentration were shown to have modulatory effect on the locomoter activity (Kayir and Uzbay, 2004). Decreased level of NO and inhibition of NO synthase were associated with increased locomotor activity (Del Bel et al., 2005; Pechánová et al., 2006). On the other hand, the NOS inhibitors have been reported to possess antidepressant-like behavioral properties as doses that are without any effect on locomotor activity (Wegener et al., 2003). NO, a messenger molecule in central nervous system (CNS), has been implicated in neurotransmission, including in depression (Esplugues, 2002). NO/cGMP (cyclic guanosine monophosphate) is reported to be involved in depression (Mantovani et al., 2003). Reduction of NO levels within the hippocampus can induce antidepressant-like effects, implicating endogenous hippocampus NO in the neurobiology of stress and depression (Joca and Guimarães, 2006). Interestingly, NO mediates both anxiolytic-like and antidepressant-like effects in animal models of anxiety and depression (Spiacci Jr. et al., 2008). Several physiological actions of NO are mediated through its interaction with the heme iron of soluble guanylate cyclase (sGC), leading to enzyme activation and consequent increase of cGMP (Kaster et al., 2005). Nowadays, it still has few reports to mention about anxiolytic-like and antidepressant-like functions generated in one molecule (Spiacci Jr. et al., 2008).

Xanthine-based compound is an o-CPP, nominated as 7-[2-[4-(2-chlorophenyl)piperazinyl]ethyl]-1,3-dimethylxanthine with a following formula I:

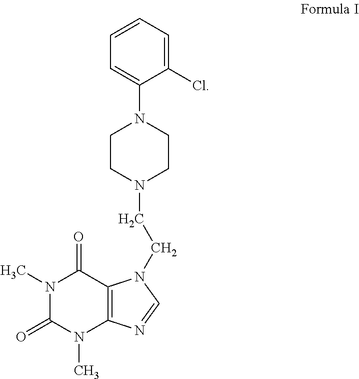

Xanthine-based compound is chemically a caffine/theophylline derivative with NO releasing, cGMP-enhancing and phosphodiesterase (PDE) inhibition activities (Wu et al., 2001; Lin et al., 2006). Xanthine-based compound increases eNOS (endothelial NOS)/sGC and decrease PDE3, PDE4 and PDE5 expressions, causing predominant accumulation of cGMP in vascular, carvenosa and tracheal smooth muscle, sharply different from non-specific cAMP (cyclic adenosine monophosphate) enhancer theophylline (Wu et al., 2001, 2004, 2005, 2006; Lin et al., 2002). Caffeine (0.5˜16 mg/kg) has been described to significantly increase locomotor activity at low dose, while low dose of NOS inhibitor L-NAME (N(G)-nitro-L-arginine methyl ester) blocked caffeine-induced locomotor activity at low dose. In addition, xanthine-based compound has been compared the cGMP-enhancing property with non-xanthine type cGMP-enhancer Sildenafil and YC-1 (Wu et al., 2006). To date, YC-1, a sGC activator with cGMP-enhancing activity, has been described to affect learning and memory activity (Monfort et al., 2001). Sildenafil, a PDE5 inhibitor also with cGMP-enhancing activity, increases the anxiety in an animal model (Riazi et al., 2006). Both YC-1 and Sildenafil mentioned the involvement of NO/cGMP in learning/memory and anxiety, respectively.

Co-localization of 5-hydroxytryptamine (5-HT) and NOS and modulation of serotonergic neurotransmission by NO has been reported (straub et al., 2007). 5-HT depletion is concomitant with change in NOS activity without affecting NOS expression. At the functional level, pharmaceutical studies have demonstrated that NO signaling can modulate 5-HT release from specific brain structures (Iuras et al., 2005). NO affects 5-HT re-uptake and appears to interact with selective 5-HT re-uptake inhibitors used in the treatment of depression. Moreover, inhibition of NOS to decrease NO production enhanced the clinical efficacy of serotonergic antidepressants (Harkin et al., 2004). This finding indicates the modulation of NO/cGMP or 5-HT by each other in anti-depression. 5-HT antagonist activity-related antidepressant activity involves the modulation of NO/cGMP.

Serotonin increased cGMP production in frontal cortical slices of rat brain. Stimulation of cGMP production was blocked by the 5-HT_{1A} receptor antagonist. Stimulation of cGMP formation by serotonin could be prevented by 5-HT_{2A}/B/c antagonist pirenperone (Regina et al., 2003). Activation of serotonin 5-HT_{1A} and 5-HT_{2A} receptors increase brain cGMP levels. Serotonin can either increase or decrease anxiety-like behavior in animals, dependent upon neuroanatonmical site of action and 5-HT receptor subtype. Although systemic studies with 5-HT2 receptor agonists and antagonists suggest a facilitated role for this receptor subtype in anxiety, somewhat inconsistent results have been obtained. Xanthine-based structure is also chemically with chlorophenyl piperazine moiety found in antidepressant Trazodone. Whether NO-releasing, cGMP-enhancing and 5-HT inhibition by xanthine-based compound displays anti-depression and sedation activity remained unidentified.

The most manifest CNS stimulation in behavior response of mice by caffeine and theophylline is locomotor activity. The most manifest pharmacologic activity of Trazodone is antidepressant activity. The present study is aimed to investigate the participation of NO/cGMP and 5-HT pathway in the antidepressant and sedation activity of xanthine-based compound, in comparison with caffeine/theophylline and Trazodone, to confirm the serotonergic modulation by NO/cGMP (Straub et al., 2007).

Because of the problems of hazard and side effect generated by the present antidepressant, antidepressant using NO and 5-HT modulation is still to be developed.

It is therefore attempted by the applicant to deal with the above situation encountered in the prior art.

### SUMMARY OF THE INVENTION

In accordance with a first aspect of the present invention, a pharmaceutical composition for treating serotonergic neurotransmission related disease or illness (especially depression) has relatively high safety and does not generate side effect on lethargy.

To achieve the above first purpose, a pharmaceutical composition for treating a serotonergic neurotransmission-related disease is provided. The pharmaceutical composition includes one of a first compound having a formula I:

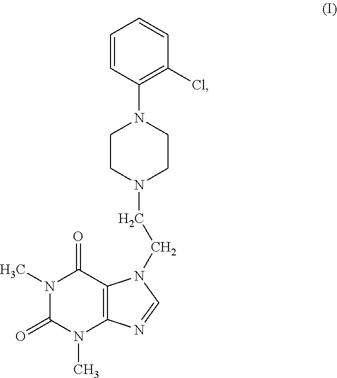

and a second compound having a formula II:

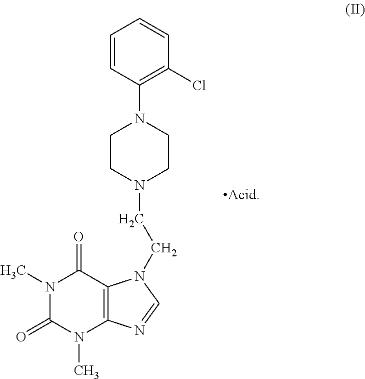

Preferably, the acid is one of an organic acid and an inorganic acid. The organic acid is one selected from a group consisting of a citric acid, a maleinic acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a pentanoic acid and an aspartic acid. The inorganic acid is one selected from a group consisting of a hydrochloride, a sulfuric acid, a phosphoric acid, a boric acid and a dihydrochloride.

One kind of the second compound has a following structural formula III when acid is replaced by hydrochloride (HCl).

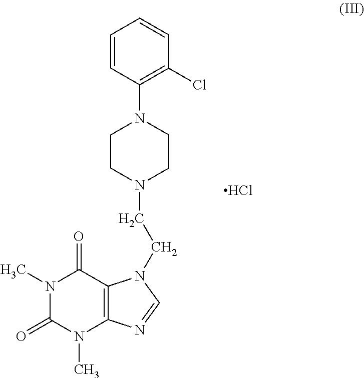

Preferably, the pharmaceutical composition further includes at least one of a pharmaceutically acceptable carrier and an excipient.

Preferably, the first compound is a 7-[2-[4-(2-chlorophenyl)-piperazinyl]ethyl]-1,3-dimethylxanthine and the second compound is a 7-[2-[4-(2-chlorophenyl)piperazinyl]ethyl]-1,3-dimethylxanthine.acid.

Preferably, the serotonergic neurotransmission is regulated through a cyclic guanosine monophosphate (cGMP) pathway.

Preferably, the disease is an illness related to a 5-hydroxytryptamine 2 (5-HT2) receptor or is a depression.

In accordance a second aspect of the present invention, a pharmaceutical composition for treating a depression is provided. The pharmaceutical composition includes one of a first compound having a formula I as above and a second compound having a formula II as above.

Preferably, the first compound has raw materials of a 2-chlorophenyl theophylline and a 2-chlorophenyl piperazine.

Preferably, the first compound has raw materials of a 7-ethylbromotheophylline and a 1-(2-chlorophenyl)piperazine.

Preferably, the pharmaceutical composition for treating the depression of a mammal is one of a human and a rodent, and the rodent is one of a mouse and a rat.

Preferably, the formula I and the formula II have a first effective amount and a second effective amount respectively, and the first effective amount and the second effective amount are ranged between 2 mg/kg of an animal body weight and 16 mg/kg of the animal body weight, respectively.

Preferably, the first effective amount and the second effective amount are ranged between 4 mg/kg of the animal body weight and 16 mg/kg of the animal body weight, respectively.

In accordance with a third aspect of the present invention, a method for treating one of a depression and a disease related to a serotonergic neurotransmission on an animal is provided. The method includes a step of administrating the animal with one of a first compound having a formula I as above and a second compound having a formula II as above.

Preferably, the mouse has an immobility time when the mouse is experienced with a forced swimming immobility test, and the first compound and a third compound cooperatively function on decreasing the immobility time.

Preferably, the third compound is one selected from a group consisting of a reserpine, an L-arginine, a methylene blue, a 7-nitroindazole, a 1-(2,5-dimethoxy-4-iodophenyl)-2-aminopropane (DOI), a meta-chlorophenyl piperazine (m-CCP) and an N-(1-methyl-1H-5-indolyl)-N′-(3-pyridinyl)urea hydrochloride (SB200646).

Preferably, the first compound and the second compound have a dosage form being at least one selected from a group consisting of an oral administration, an intravenous injection, an subcutaneous injection, an intraperitoneal injection, an intramuscular injection and a sublingual administration.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 illustrates a bar chart showing the effect of different doses (4, 8 and 16 mg/kg, respectively) of Pulmodil on mean immobility time in the mouse forced swim test (FST);

FIG. 2 illustrates a bar chart showing the effect of the compounds (Sildenafil, Trazodone and Pulmodil, 4 mg/kg each) on mean immobility time in the FST;

FIG. 3 illustrates a bar chart showing the effect of mean immobility time in the FST, wherein mice are pre-treated with Reserpine (2 mg/kg) and then treated with Pulmodil (2, 3, 4 and 8 mg/kg, respectively);

FIG. 4 illustrates a bar chart showing the effect of mean immobility time in the FST, wherein mice are pre-treated with Reserpine (2 mg/kg) and then treated with Pulmodil, Sildenafil and Trazodone (4 mg/kg), respectively;

FIG. 5 illustrates a bar chart showing the effect of mean immobility time in the FST, wherein mice are merely given L-arginine (750 mg/kg, intraperitoneal (i.p.) injection) or are given L-arginine (750 mg/kg, i.p.) for 30 minutes and then Pulmodil (16 mg/kg, i.p.);

FIG. 6 illustrates a bar chart showing the effect of mean immobility time in the FST, wherein mice are given methylene blue (10 mg/kg, i.p.) for 30 minutes and then Pulmodil (4 or 8 mg/kg, i.p.);

FIG. 7 illustrates a bar chart showing the effect of mean immobility time in the FST, wherein mice are respectively given Pulmodil (8 mg/kg, i.p.), 7-nitroindazole (25 mg/kg i.p.), and 7-nitroindazole (25 mg/kg, i.p.) for 30 minutes and then Pulmodil (8 mg/kg, i.p.);

FIG. 8 illustrates a bar chart showing the effect of mean immobility time in the FST, wherein mice are merely given DOI (4 mg/kg, i.p.), and given DOI (4 mg/kg, i.p.) for 30 minutes and Pulmodil (4 or 8 mg/kg, i.p.);

FIG. 9 illustrates a bar chart showing the effect of mean immobility time in the FST, wherein mice are given m-CPP (4 mg/kg, i.p.) and then Pulmodil (4 or 8 mg/kg, i.p.);

FIG. 10 illustrates a bar chart showing the effect of mean immobility time in the FST, wherein mice are given SB200646 (4 mg/kg, i.p.) and then Pulmodil (4 or 8 mg/kg, i.p.); and

FIG. 11 illustrates a bar chart showing the comparison of different doses (0.5, 1 and 2 mg/kg, respectively) of Pulmodil on cGMP concentration of mouse brain.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

As illustrated above, xanthine-based compound is nominated as 7-[2-[4-(2-chlorophenyl)piperazinyl]ethyl]-1,3-dimethylxanthine which has a structural formula (Formula I) as follows.

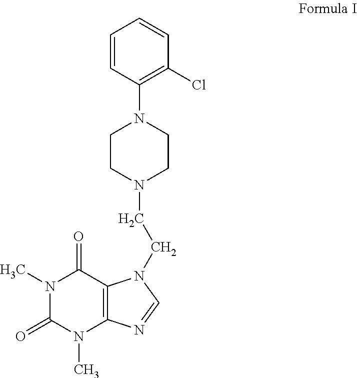

Among these, 2-chlorophenyl piperazine group is the aforementioned o-CPP, which regulates serotonergic neurotransmission and NO/cGMP pathway. In addition, xanthine-based compound does not generate lethargy effect per se since o-CPP lacks anti-histaminergic activity.

When animals are stimulated (such as psychological stress) or has abnormal physiological conditions to influence the original serotonergic neurotransmission, they might induce various relevant psychological or physiological disease or illness including 5-HT2 receptor regulation-related disease or illness (depression as a typical example).

To treat serotonergic neurotransmission-related disease or illness, chlorophenyl piperazine derivative or its pharmaceutically acceptable salt derivatives are provided in the present invention.

The preferred pharmaceutically acceptable salt derivatives are organic salts or inorganic salts of chlorophenyl piperazine derivative. The organic salt ones are formed from organic acids including citric acid, maleinic acid, fumaric acid, tartaric acid, oleic acid, stearic acid, benzenesulphonic acid, ethyl benzenesulphonic acid, benzoic acid, succinic acid, mesylic acid, dimesylic acid, acetic acid, propionic acid, pentanoic acid and aspartic acid. The inorganic salt ones are formed from inorganic acids including hydrochloride, sulfuric acid, phosphoric acid, boric acid and dihydrochloride.

The best pharmaceutically acceptable salt derivative is hydrochloride (HCl) of chlorophenyl piperazine derivative nominated as Pulmodil having a following structure of Formula III:

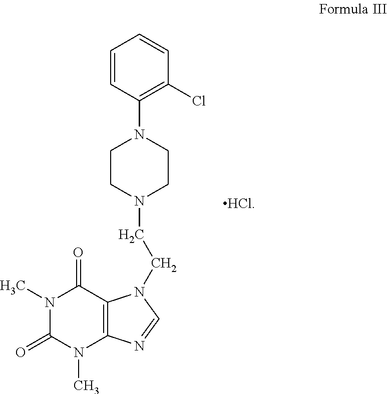

Chlorophenyl piperazine derivative or the pharmaceutically acceptable salts can be mixed with pharmaceutically acceptable carrier or excipient, and various mammals are administrated with oral format (such as tablet and capsule) or with non-oral format (such as intravenous, subcutaneous, intraperitoneal, intramuscular and sublingual administrations). The preferred mammals are rodents or humans, and the best one is humans.

The skilled person can determine the effective amount (i.e. the amount for reducing patients' symptoms or illness) of chlorophenyl piperazine derivative or the pharmaceutically acceptable salts depending on the patients' disease seriousness and physiological conditions so as to administrate drugs, perform serotonergic neurotransmission regulation (particularly antidepressant effect).

Generally speaking, a dosing amount of active chlorophenyl piperazine derivative or pharmaceutically acceptable salts is 2˜16 mg/kg, and the preferred one thereof is 4˜16 mg/kg. However, this dosing range can be adjusted over the above-mentioned reference dosing range due to the difference of healthy level, tolerance level or disease/illness development of respective mammals.

### Biological Experiments

For proving antidepressant effect of the chlorophenyl piperazine derivative (formula I) or its pharmaceutically acceptable salt derivative (formula II) on patients, hydrochloride salt (i.e. Pulmodil, formula I) of chlorophenyl piperazine derivative was administrated in the mouse model and was compared with other NO/cGMP or 5-HT regulation-related reagents, so as to prove that the antidepressant effect is achieved by NO/cGMP or 5-HT regulation.

Materials and Methods

1. Animal Source:

Male ddk mice weighing between 22 and 30 g, bred in the Center Animal House facility of Kaohsiung Medical University (KMU), Taiwan, were used. These experimental animals were housed under standard laboratory conditions and maintained on natural light and dark cycle, and had free access to food and water. Animals were acclimatized to laboratory conditions before the experiment. Each animal was used only once. All the experiments were carried out between 9 a.m. and 3 p.m. The experimental protocols were approved by the Animal Center of KMU, Taiwan.

2. Forced Swim Test (FST):

Forced swim would induce immobility of an animal to simulate the phenomenon of human depression (Renard et al., 2003) and antidepressant was used to reverse this phenomenon (Kulkarni and Mehta, 1985). This test procedure was carried out according to the previous animal model (Reddy et al., 1998). In brief, mice were individually forced to swim inside a visual plastic cylinder (diameter: 9 cm, containing 15 cm of water maintained at 23˜25° C. After the initial 2˜3 minutes of vigorous activity, mice showed period of immobility by floating with minimum movements. An animal was considered to be immobile whenever it remained floating in the water in a slightly hunched but upright position, its nose above the water surface. The total immobility time for the period of 6 minutes was recorded with the help of a stop-watch.

3. cGMP Analysis of Mouse Brain:

Male mice were given Pulmodil with intraperitoneal (i.p.) injection, where mice were divided into three groups, and 0.5, 1 and 2 mg/kg Pulmodil respectively for each group. After 30-minute injection, mice were anesthetized with 4,000 mg/kg chloral hydrate, and then were fixed on the stereotaxic frame (Narishige, Japan) to obtain mice brain. The isolated brain was homogenized and extracted with 0.1 N HCl. The extract was centrifuged at 4° C., at 14,000 rpm for 30 minutes, and supernatant was collected to determine cGMP concentration, which was determined with commercial enzyme-linked immunosorbent assay (ELISA) kit (Assay Designs) by acetylation procedure.

4. Preparation of Drugs:

The following chemicals were used in the present invention, including Pulmodil (Syn-tech Chem. & Pharm. Co., Ltd., Taiwan), Sildenafil (Panacea Biotec, New Delhi, India), Trazodone, Reserpine, L-arginine (Sigma Co.), methylene blue (Sigma Co.), 7-nitroindazole (Tocris Bioscience, MO, U.S.A.), DOI (1-(2,5-dimethoxy-4-iodophenyl)-2-aminopropane), meta-chlorophenylpiperazine (m-CPP) and SB200646 (N-(1-methyl-1H-5-indolyl)-N′-(3-pyridinyl)urea hydrochloride). All the drugs were dissolved in 0.9% (w/v) NaCl except 7-nitroindazole which was dissolved in a few drops of Tween 80 and the volume was made with distilled water (0.9% (w/v)).

The doses of the drugs used were selected according to the previous studies (Patil et al., 2005). Different doses were administrated intraperitoneally in a fixed volume of 1 ml/100 g body weight for 30 minutes before the animals were subjected to test.

The possible participation of the NO/cGMP pathway in the antidepressant effect of Pulmodil was investigated. Mice were pretreated with NO/cGMP pathway regulation related drugs (Reserpine, L-arginine, methylene blue or 7-nitroindazole), and then Pulmodil or vehicle was injected to the animal. Forced swim test was performed 30 minutes later.

The possible participation of the 5-HT pathway in the antidepressant effect of Pulmodul was investigated. Mice were pretreated with 5-HT pathway related drugs (DOI, m-CPP or SB200646), and then Pulmodil or vehicle was injected to the animal. Forced swim test was performed 30 minutes later.

5. Statistical Analysis:

Results expressed as mean (sec.)±S.E.M. and the data was analyzed using One-Way or Two-Way Analysis of Variance (ANOVA) where appropriate. If any statistically significant change was found, post-hoc comparisons were performed using a Dunnett's test. Data was deemed significant when P<0.05 was considered statistically significant.

Results

1. Effect on Forced Swimming Immobility by Pulmodil:

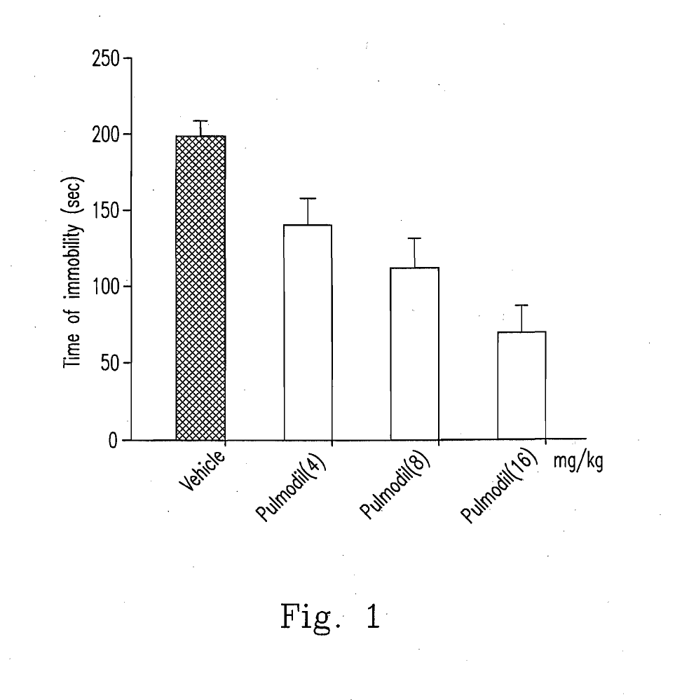

Please refer to FIG. 1, which illustrates a bar chart showing the effect of different doses (4, 8 and 16 mg/kg, respectively) of Pulmodil on mean immobility time in the mouse FST. Compared with vehicle, 4, 8 and 16 mg/kg of Pulmodil respectively produced a decrease in immobility period (p<0.05).

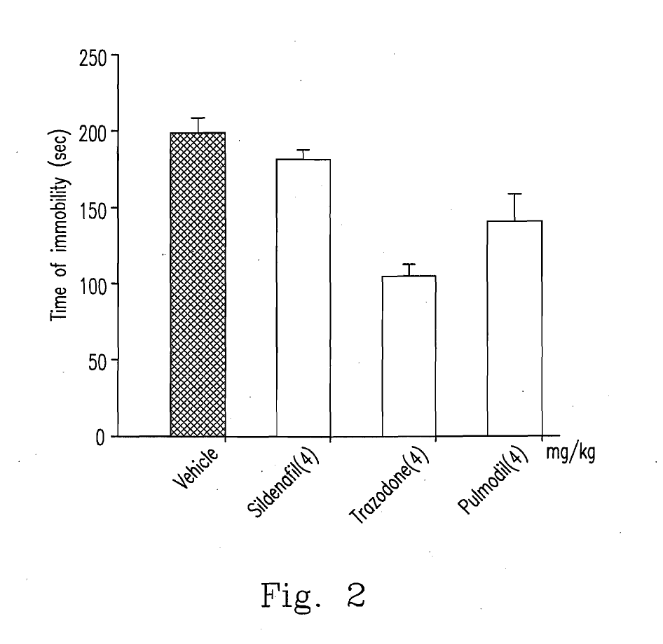

Please refer to FIG. 2, which illustrates a bar chart showing the effect of the compounds (Sildenafil, Trazodone and Pulmodil, 4 mg/kg each) on mean immobility time in the FST. In comparison, Trazodone, being the antidepressant in the prior art, significantly decreased the immobility period (p<0.05) of mice, but Sildenafil could not significantly affect immobility time.

2. Effect on Reserpine-Induced Forced Swimming Immobility:

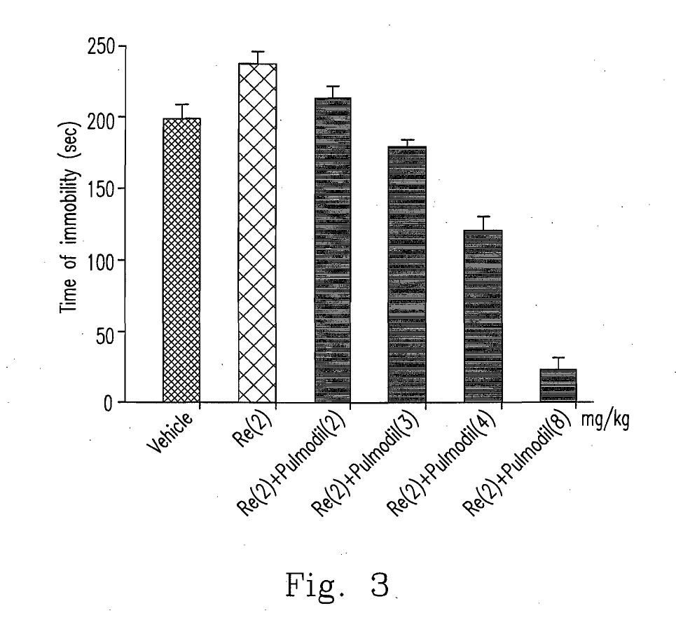

Please refer to FIG. 3, which illustrates a bar chart showing the effect of mean immobility time in the FST, wherein mice are pre-treated with Reserpine (2 mg/kg) and then treated with Pulmodil (2, 3, 4 and 8 mg/kg, respectively). The abbreviation, “Re”, was mentioned as Reserpine.

Reserpine is a hypertension drug which also shows sedation. However, patients occasionally feel depressed after dosing Reserpine. Reserpine increased immobility period (p<0.05) in mouse FST. Pre-treatment of mice with Reserpine (2 mg/kg) for 4 hours, xanthine-based compound (2, 3, 4 or 8 mg/kg), dose-dependently reduced the immobility period of mice or caused antidepressant activity in mice (p<0.05, significantly different from Reserpine), compared to Reserpine without xanthine-based compound.

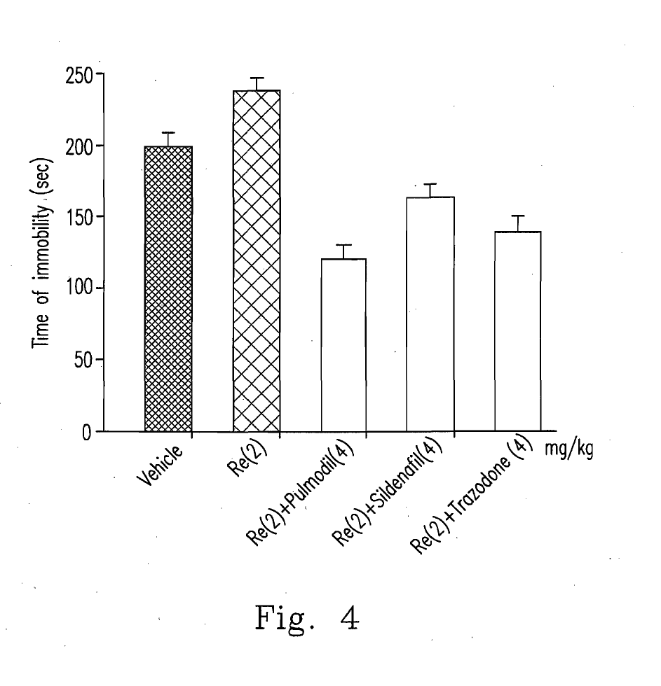

Please refer to FIG. 4, which illustrates a bar chart showing the effect of mean immobility time in the FST, wherein mice are pre-treated with Reserpine (2 mg/kg) and then treated with Pulmodil, Sildenafil and Trazodone (4 mg/kg), respectively. In comparison, Reserpine-induced immobility was significantly inhibited by xanthine-based compound, Sildenafil and Trazodone (p<0.05), respectively.

3. Reversal by L-Arginine in Forced Swimming Immobility

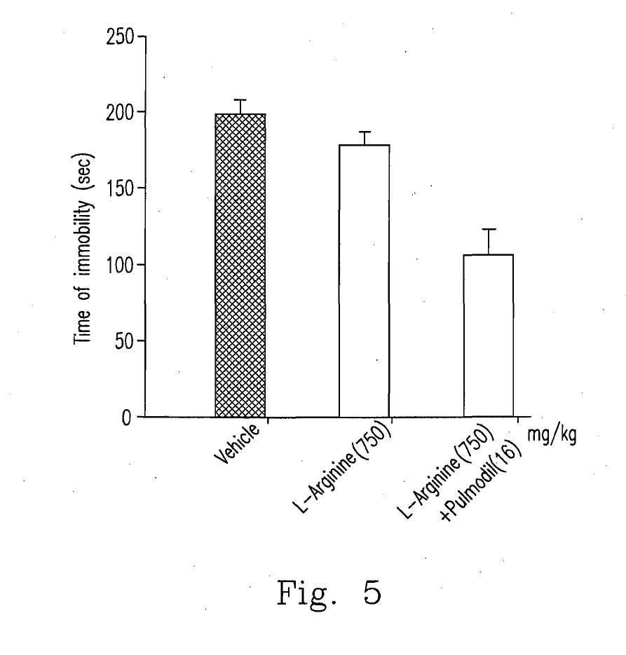

Please refer to FIG. 5, which illustrates a bar chart showing the effect of mean immobility time in the FST, wherein mice are merely given L-arginine (750 mg/kg, intraperitoneal (i.p.) injection) or are given L-arginine (750 mg/kg, i.p.) for 30 minutes and then Pulmodil (16 mg/kg, i.p.). L-arginine is a NO precursor. This experiment was found that i.p. administration with an effective dose of L-arginine inhibited the antidepressant action of Pulmodil (p<0.05, significantly different from the vehicle) as shown by an increase in immobility period compared to Pulmodil without L-arginine (FIG. 1).

4. Enhanced by sGC Inhibitor in Forced Swimming Immobility

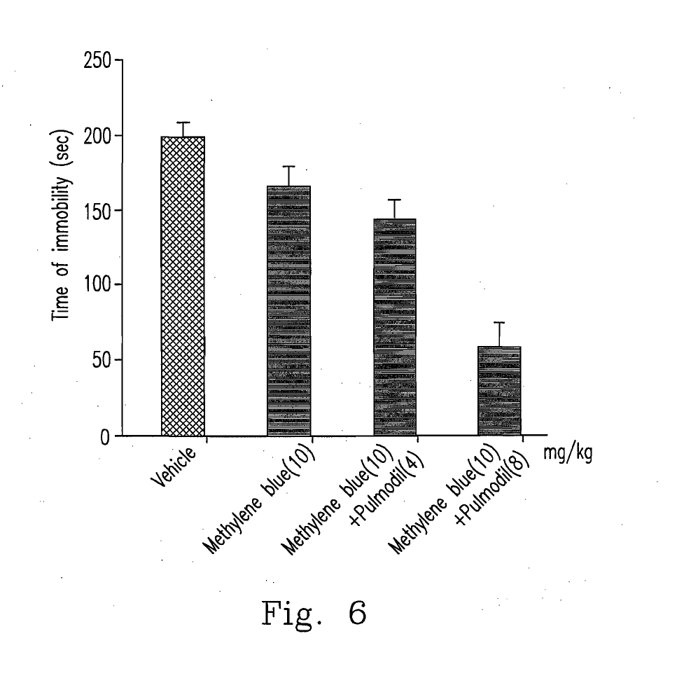

Please refer to FIG. 6, which illustrates a bar chart showing the effect of mean immobility time in the FST, wherein mice are given methylene blue (10 mg/kg, i.p.) for 30 minutes and then Pulmodil (4 or 8 mg/kg, i.p.). Methylene blue, a direct inhibitor of both NOS and sGC, did not affect the immobility time per se. However, methylene blue significantly decreased the immobility time (p<0.05, significantly different from the vehicle) of Pulmodil (8 mg/kg, i.p.) or enhanced the antidepressant effect of Pulmodil (8 mg/kg, i.p.).

5. Enhanced by nNOS Inhibitor in Forced Swimming Immobility

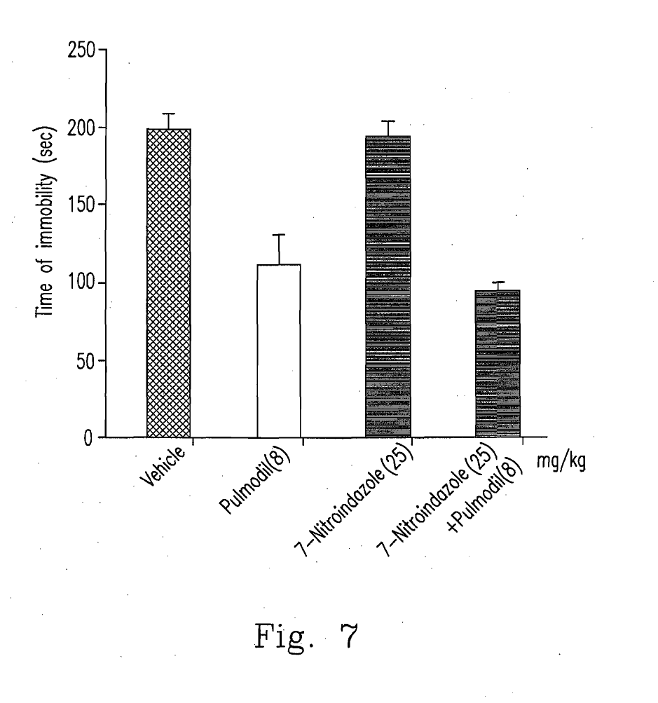

Please refer to FIG. 7, which illustrates a bar chart showing the effect of mean immobility time in the FST, wherein mice are respectively given Pulmodil (8 mg/kg, i.p.), 7-nitroindazole (25 mg/kg i.p.), and 7-nitroindazole (25 mg/kg, i.p.) for 30 minutes and then Pulmodil (8 mg/kg, i.p.). 7-Nitroindazole, a specific nNOS inhibitor, shortened the immobility time or enhanced the antidepressant effect of Pulmodil (P<0.05).

6. Reversal by 5-HT Agonist in Forced Swimming Immobility

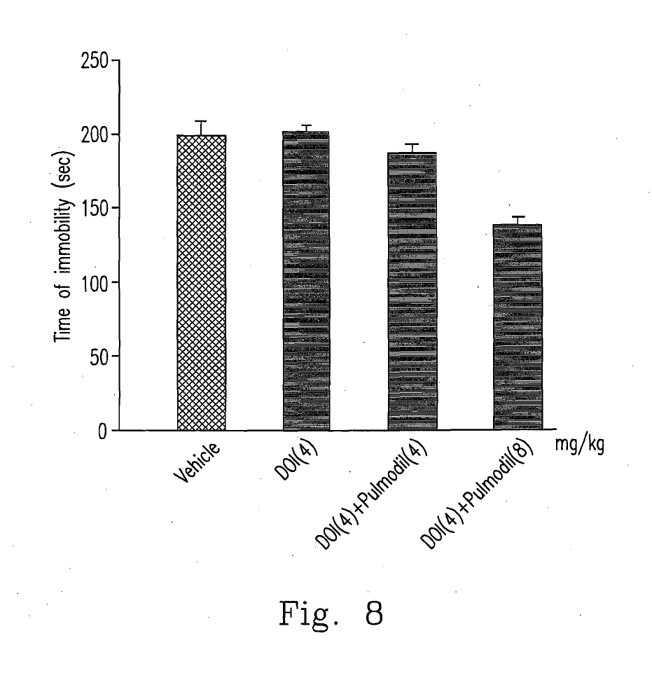

Please refer to FIG. 8, which illustrates a bar chart showing the effect of mean immobility time in the FST, wherein mice are merely given DOI (4 mg/kg, i.p.), and given DOI (4 mg/kg, i.p.) for 30 minutes and Pulmodil (4 or 8 mg/kg, i.p.). DOI, a 5-HT agonist, did not affect the immobility of mice; however, in comparison with FIG. 1, pre-treatment with DOI prolonged the immobility time or reduced the antidepressant activity of Pulmodil (p<0.05).

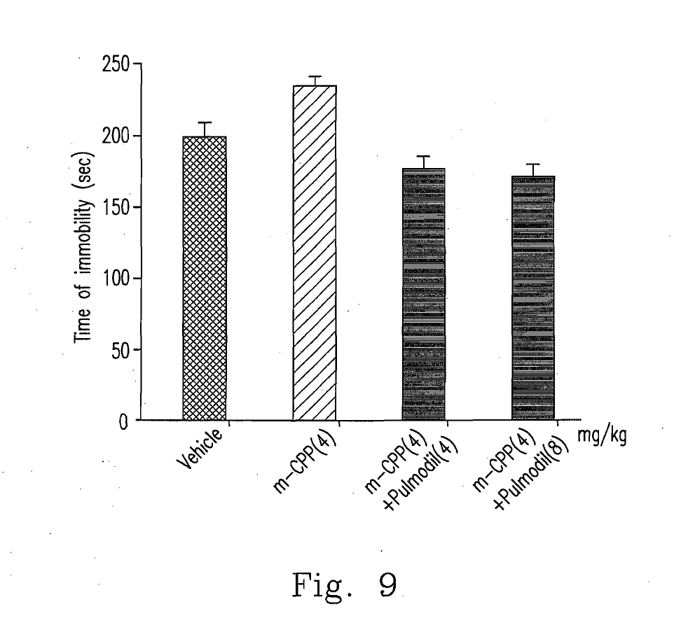

Please refer to FIG. 9, which illustrates a bar chart showing the effect of mean immobility time in the FST, wherein mice are given m-CPP (4 mg/kg, i.p.) and then Pulmodil (4 or 8 mg/kg, i.p.). m-CPP, also a 5-HT agonist, mildly increased the immobility time per se (p<0.05, significantly different with the vehicle). In comparison with FIG. 1, pre-treatment with m-CPP shortened this immobility time or reversed the antidepressant activity of Pulmodil (p<0.05).

7. Enhanced by 5-HT Antagonist in Forced Swimming Immobility

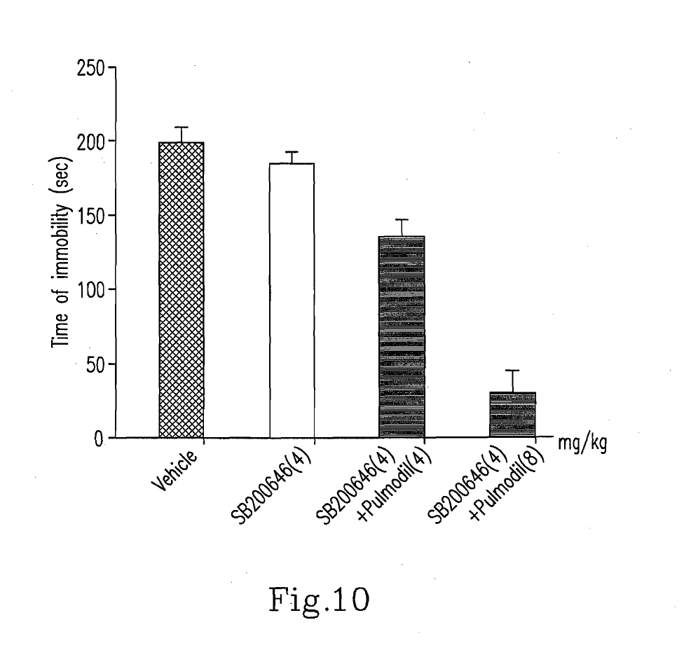

Please refer to FIG. 10, which illustrates a bar chart showing the effect of mean immobility time in the FST, wherein mice are given SB200646 (4 mg/kg, i.p.) and then Pulmodil (4 or 8 mg/kg, i.p.). SB200646, a 5-HT antagonist, did not affect the immobility time of mice per se. However, pre-treatment with SB200646 significantly reduced this immobility time or enhanced the antidepressant activity of Pulmodil (p<0.05).

8. Promoted cGMP Concentration in Mouse Brain by Pulmodil

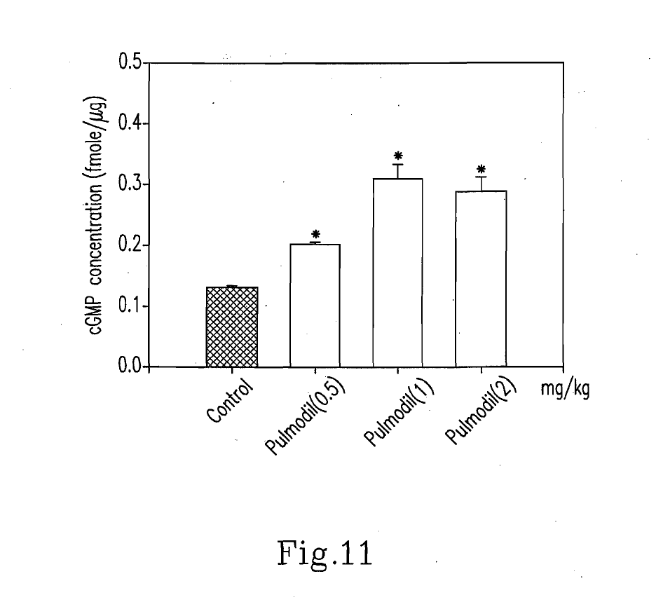

Please refer to FIG. 11, which illustrates a bar chart showing the comparison of different doses (0.5, 1 and 2 mg/kg, respectively) of Pulmodil on cGMP concentration of mouse brain. The basal concentration of cGMP in mouse brain was 0.131±0.003 fmol/μg (vehicle), and cGMP concentration in the mouse brain would be significantly increased due to i.p. injection of Pulmodil (0.5, 1 and 2 mg/kg, respectively). cGMP concentration in the brain was dose-independently increased as 0.201±0.003, 0.293±0.023 and 0.288±0.0234 fmol/μg, respectively, post Pulmodil injection.

It is known from the Pulmodil-involved results that Pulmodil and the pharmaceutically acceptable salt derivatives (such as hydrochloric salt derivative, citric salt derivative and so on) can achieve anti-depression and desation by 5-HT2 receptor antagonism and NO/cGMP-related regulation. Further, Pulmodil and the pharmaceutically acceptable salt derivatives do not have anti-histaminergic activity. Thus, Pulmodil and the pharmaceutically acceptable salt derivatives are adequately applied in preparation of pharmaceutical composition or drugs so as to treat serotonergic neurotransmission-related diseases or illness, particularly applied in antidepressant without side effect on lethargy.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A method for treating one of a depression and a disease related to a serotonergic neurotransmission on an animal, the method comprising a step of administrating the animal with one of a first compound having a formula I: 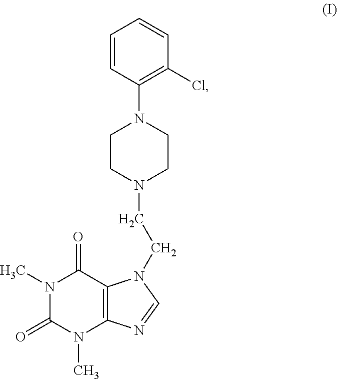
and a second compound having a formula II:

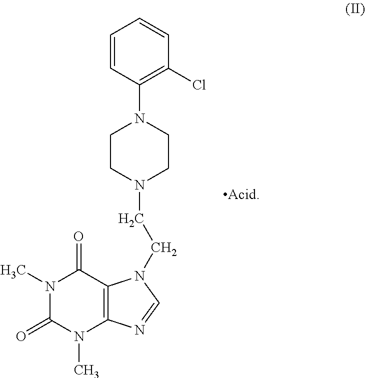

2. The method according to claim 1, wherein the mouse has an immobility time when the mouse is experienced with a forced swimming immobility test, and the first compound and a third compound cooperatively function on decreasing the immobility time.

3. The method according to claim 2, wherein the third compound is one selected from a group consisting of a reserpine, an L-arginine, a methylene blue, a 7-nitroindazole, a 1-(2,5-dimethoxy-4-iodophenyl)-2-aminopropane (DOI), a meta-chlorophenyl piperazine (m-CCP) and an N-(1-methyl-1H-5-indolyl)-N′-(3-pyridinyl)urea hydrochloride (SB200646).

4. The method according to claim 1, wherein the first compound and the second compound have a dosage form being at least one selected from a group consisting of an oral administration, an intravenous injection, an subcutaneous injection, an intraperitoneal injection, an intramuscular injection and a sublingual administration.
